(19) United States
(12) Patent Application Publication
(10) Pub. No.: US 2019/0145945 A1
(43) Pub. Date: May 16, 2019

(54) ELECTROCHEMICAL GAS SENSOR FOR USE IN ULTRA LOW OXYGEN STORAGE ENVIRONMENTS

(71) Applicant: HONEYWELL INTERNATIONAL INC., Morris Plains, NJ (US)

(72) Inventor: Steve P. GAUTIERI, Morris Plains, NJ (US)

(21) Appl. No.: 16/307,166

(22) PCT Filed: May 26, 2017
(86) PCT No.: PCT/US2017/034733

§ 371 Date: Dec. 5, 2018

Related U.S. Application Data
(60) Provisional application No. 62/345,944, filed on Jun. 6, 2016.

(51) Int. Cl. G01N 33/00; F25D 29/00; G01N 27/416; G01N 27/403; G08B 21/14

(52) U.S. Cl. CPC G01N 33/0018; G01N 33/0054; G08B 21/14; G01N 27/4163; G01N 27/403; F25D 29/005

(57) ABSTRACT

Embodiments include methods and systems for providing gas detection for a low oxygen storage room. A gas sensor system may comprise a first air flow conduit from the interior of a low oxygen storage room; a second air flow conduit from the exterior of the low oxygen storage room, wherein the oxygen content of a second air flow through the second air flow conduit is greater than an oxygen content of a first air flow through the first air flow conduit; a mixing joint joining the first air flow conduit with the second air flow conduit and in fluid communication with a joined air flow conduit; and a gas sensor configured to receive a joined air flow through the joined air flow conduit and configured to detect ammonia levels in the joined air flow, wherein the gas sensor is calibrated for the flow rates of the two air flows.

### CROSS-REFERENCE TO RELATED APPLICATIONS

The present application claims priority to U.S. Provisional Patent Application Ser. No. 62,345,944 filed Jun. 6, 2016 by Steve P. Gautieri and entitled “Electrochemical Gas Sensor for Use in Ultra Low Oxygen Storage Environments” which is incorporated herein by reference as if reproduced in its entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Sensors are commonly used to sense environmental parameters such as pressure, temperature, humidity, flow,thermal conductivity, gas concentration; light, magnetic fields, electric fields, as well as many other environmental parameters. Such sensors are used in a wide variety of applications including, for example, medical applications, flight control applications, industrial process applications combustion control applications, weather monitoring applications, water metering applications, as well as many other applications.

### SUMMARY

In an embodiment, a gas sensor system may comprise a first air flow conduit from the interior of a low oxygen storage room; a second air flow conduit from the exterior of the low oxygen storage room, wherein the oxygen content of a second air flow through the second air flow conduit is greater than an oxygen content of a first air flow through the first air flow conduit; a mixing joint joining the first air flow conduit with the second air flow conduit and in fluid communication with a joined air flow conduit; and a gas sensor configured to receive a joined air flow through the joined air flow conduit and configured to detect ammonia levels in the joined air flow, wherein the gas sensor is calibrated for the flow rates of the two air flows.

In an embodiment, a method for providing gas detection for a low oxygen storage room may comprise drawing a first air flow from the interior of the low oxygen storage room; drawing a second air flow from the exterior of the low oxygen storage room, wherein the oxygen content of the second air flow is greater than the oxygen content of the first air flow; monitoring the flow rates of the first air flow and the second air flow; joining the first air flow and the second air flow; feeding the joined air flows to a gas sensor; and detecting the gas content of the combined air flows, wherein the detection comprises a reduction-oxidation reaction.

In an embodiment, a method for providing gas detection for a control led atmosphere storage room may comprise drawing a first air flow from the interior of the low atmosphere storage room; drawing a second air flow from the exterior of the low atmosphere storage room, wherein the oxygen content of the second air flow is greater than the oxygen content of the first air flow; monitoring the flow rates of the first air flow and the second air flow; joining the first air flow and the second air flow; feeding the joined air flows to a gas sensor; detecting the gas content of the combined air flows, wherein the detection comprises a reduction-oxidation reaction; and in response to detecting the gas content of the combined air flows, determining the gas content of the interior of the low atmosphere storage room using the monitored flow rates for the first air flow and the second air flow.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a gas sensor system for use with controlled atmosphere storage according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Embodiments of the disclosure include systems and methods for providing a gas sensor for use with controlled atmosphere storage, particularly systems using ammonia(NH₃) as a refrigerant. Typical controlled atmosphere (CA) storage of food and pharmaceutical products may not include sufficient gas detection systems for when ammonia is used as a refrigerant. Typical solutions may not provide a low-cost, low-level ammonia detection system for these applications.

CA storage is a technique where the level of oxygen is reduced in a gas-tight environment for the storage of sensitive pharmaceutical products, fruit, and vegetables inhibiting the ripening and ageing processes, thus retaining flavor and quality. The concentrations of oxygen, carbon dioxide and nitrogen, as well as the temperature and humidity of the storage room are regulated. Both dry commodities and fresh fruit and vegetables can be stored in controlled atmospheres. The combination of altered atmospheric conditions and reduced temperature allow prolonged storage with only a slow loss of quality.

The long-term storage of vegetables and fruit involves inhibiting the ripening and ageing processes, thus retaining flavor and quality. Ripening is delayed by modifying the atmospheric gas concentrations (such as reducing the level of oxygen and increasing that of carbon dioxide) in the gas-tight environment so that the respiration of fruit and vegetables is reduced. Under controlled atmosphere conditions, the quality and the freshness of fruit and vegetables are retained without the use of unnecessary chemicals. Under CA conditions, many products can be stored for 3 to 6 times longer than usual, in some cases up to a year.

Ammonia is commonly used as a refrigerant to cool CA storage facilities. Because of ammonia's vaporization properties, it is a useful refrigerant. Ammonia is a preferred low-cost environmentally friendly refrigerant used for most industrial food processes, cold storage, and pharmaceutical applications. Ammonia is a very desirable refrigerant, as its Global Warming Potential (GWP) is zero because of its very low narrow infrared (TR) absorption characteristics. Ammonia is abundantly available, found all throughout nature, and extremely efficient in refrigeration systems requiring less energy to be utilized per British Thermal Unit (BTU) of cooling output.

However, ammonia suffers from the disadvantage of toxicity, which restricts its domestic and small-scale use. Along with its use in modem vapor-compression refrigeration it is used in a mixture along with hydrogen and water in absorption refrigerators. Although common in nature and in wide use, ammonia is both caustic and hazardous in its concentrated form. It is classified as an extremely hazardous substance. The U.S. Occupational Safety and Health Administration (OSHA) has set a 15-minute exposure limit for gaseous ammonia of 35 ppm by volume in the environmental air and an 8-hour exposure limit of 25 ppm by volume.

Electrochemical gas sensors are currently the preferred type of sensing technology used for ammonia gas detection. Electrochemistry can economically detect low-level concentrations of ammonia easier than optical techniques. Examples of optical gas sensors such as non-dispersive infrared (NDIR), photoacoustic, etc. are costly in nature compared to electrochemical when configured to detect ammonia at low concentrations below 100 ppm. Low-cost ammonia optical gas sensors do not exist for low concentrations, because ammonia has very weak IR absorption characteristics requiring high-gain sensitive components, highly polished surfaces, long path lengths, and complex precision optics. Ammonia has a very low absorption characteristic at this wavelength. In general, NDIR ammonia detectors are not practical, as the main ammonia absorption bands at 3.3 μm, 10.4 μm , and 10.75 μm are very narrow and weak, making it difficult to achieve stable signal levels for accurate detection readings near the 0-100 ppm range as the zero has a tendency to drift over time with this type of sensing technology.

Ammonia detection is needed (and may be mandatory) to ensure human safety, as inhalation of ammonia may be toxic and deadly. Additionally, ingestion of food contaminated by ammonia may be toxic and deadly, should an unknown low-level ammonia leak occur within a food storage facility. Ammonia has a high affinity for water and can migrate through materials, such as membranes, plastics, and cardboard, and combine with moisture in foods, creating toxic ammonium hydroxide and destroying the exposed food. The alarm threshold for ammonia may typically be approximately 25 ppm. There are numerous codes including IIAR-2A, ASHRAE 15, NFPA-1, UMC, IFC, and IMC that regulate ammonia levels. In addition to these codes, many insurance carriers may impose their own requirements to mitigate the risk of loss of life and product in a facility.

Electrochemical sensors react with ammonia when oxygen is present to complete a re-dox reaction that generates free electrons within an electrochemical cell inside the gas sensor. The gas sensor converts the free electrons to a milliamp signal level output which is directly proportional to the ammonia concentration surrounding the gas sensor. To provide sufficient oxygen content in the sample for the sensor to function properly, additional elements may be added to the sensor system.

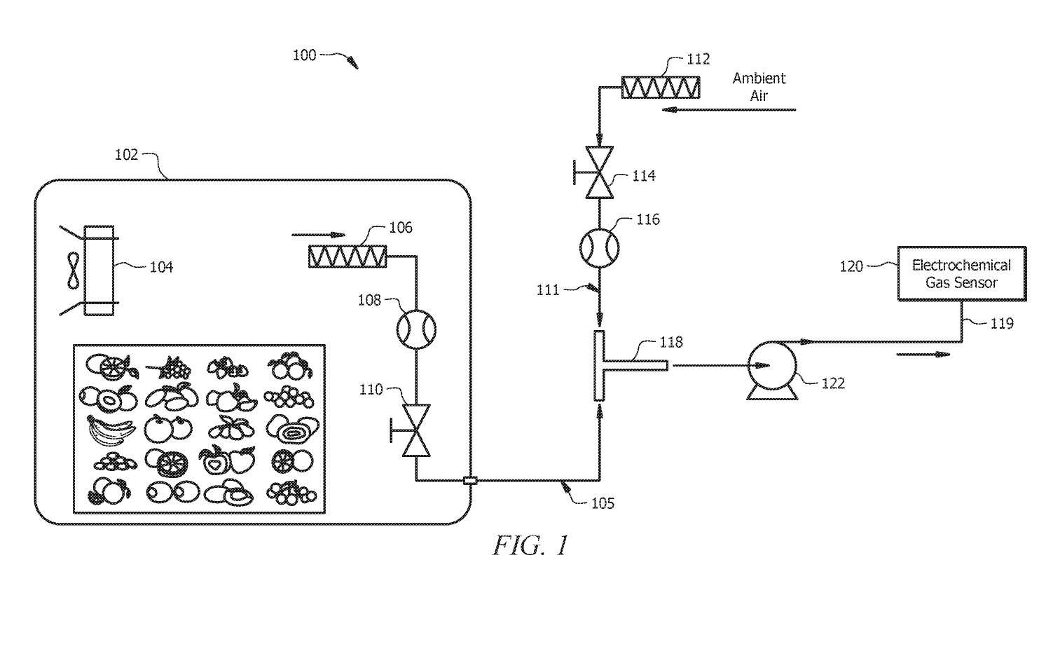

FIG. 1 illustrates a diagram of a gas sensor system 100 for a low oxygen sealed storage room 102. The storage room 102 may comprise an ammonia evaporator cooling coil 104 located within the room 102, configured to control the temperature of the sealed room 102. For example, the sealed storage room 102 may contain food products that are preserved at a low temperature. Additionally, the sealed storage room 102 may comprise a low oxygen environment.

When operating an ammonia evaporator, it may be desired to sample the air of the storage room 102 for ammonia levels present within the room 102. In FIG. 1, the air from the room 102 may be drawn from the room 102 to a gas sensor 120, wherein the air flow via conduit 105 from the room 102 may pass through a particulate air filter 106 and a flow meter 108. Additionally, the air flow via the conduit 105 from the room 102 may be controlled with a variable screw valve 110. In one embodiment of the system 100, the air filter 106, flow meter 108, and valve 110 may be located on the interior of the sealed storage room 102. In other embodiments of the system 100, one or more of the air filter 106, flow meter 108 and/or valve 110 may be located on the exterior of the sealed storage room 102.

The air flow via the conduit 105 from the room 102 may be pulled toward the gas sensor 120 with an air pump 122. In an embodiment, the air pump 122 may comprise a vacuum pump. The gas sensor 120 may receive the air flow from the conduit 105 from the room 102 and detect the level of ammonia (and possibly other characteristics of the air sample). In an embodiment, the gas sensor 120 may be located on the exterior of the room 102 where the gas sensor 120 may operate near room temperature (or approximately 72° F.), which may improve the functioning of the sensor in some instances.

In the embodiment of the system 100 shown in FIG. 1, the air flow conduit 105 from the sealed storage room 102 may be combined with another air flow conduit ill before it reaches the gas sensor 120. In order to provide a higher oxygen (O₂) content to the air sample delivered by the joined air flow conduit 119 to the gas sensor 120, an O₂ rich air flow conduit 111 located outside of the storage room 102 may be added to the air flow conduit 105 from the storage room 102. The two air flow conduits 105 and 111 may be combined with a mixing point such as a T joint 118. In an embodiment of the system, the O₂ rich air flow conduit 111 may pass through a particulate air filter 112 and a flow meter 116. Additionally, the air flow conduit 105 from the storage room 102 may be controlled with a variable air flow valve 114.

In an embodiment, the flow rate of the two air flow conduits 105 and 111 may be independently controlled with the valves 110 and 114, and may be monitored via the flow meters 108 and 116. In an embodiment, joined air flow conduit 119 may comprise approximately 70% from the first air flow conduit 105 and 30% from the second air flow conduit 111. In an embodiment, the joined air flow conduit 119 may comprise an oxygen content between approximately 5-10%.

The system 100 shown in FIG. 1 allows an electrochemical sensor to be used with an O₂ depleted sample gas by using a sample draw air vacuum pump 122 bringing in a metered fresh air flow conduit 111 outside the sealed CA storage room 102, which has ambient O₂ levels of approximately 20%, and then mixing it with a metered air flow conduit 105 of air inside the sealed storage room 102, which has depleted O₂. The two air flow conduits 105 and 111 are then mixed and fed into the electrochemical gas sensor 120. The outside ambient air flow conduit 111 has sufficient O₂ levels to feed the reduction-oxidation (redox) reaction that can accurately sense ammonia if a leak occurs within the refrigerant system 104. Each flow tube is set to a fixed metered flow at initial setup by adjusting air flow valves 110, 114 to achieve a balanced flow from each tube before it is fed through the mixing “T” joint 118.

The gas sensor 120 may be calibrated by placing a calibration adapter that seals around the CA storage sample draw particulate filter air 106. This calibration adapter has a calibration gas input port and a relief exhaust port. The calibration gas in the adapter must flow more than the metered flow in the CA storage so excess calibration gas exits the exhaust port. This is necessary so the gas drawn into the input particulate filter by the sample draw air vacuum pump 122 is 100% calibration gas without changing the pressure or flow rate or drawing in CA room ambient gas. A pressure change may be avoided in order to avoid changing the relative mixing ratio at the downstream mixing point. After the calibration gas is flowing in the CA room tube, the concentration is diluted at the gas sensor because the sample tube from outside the CA room is mixed at the mixing point.

In some embodiments, the span on the gas sensor can be set to a full-scale output based on the calibration gas assuming the calibration gas applied in the CA room has full-scale concentration. This span setting intrinsically adjusts for the diluted calibration gas reaching the sensor as ambient air flow not containing calibration gas outside the CA room is mixed before entering the gas sensor. This system act to ammonia levels such that if full-scale ammonia contaminated air exists inside the CA room, the sensor will output full-scale signals or normalize to the calibration span as the clean air flow from outside the CA room was mixed with calibration gas at the same flow rate.

The gas sensor 120 may comprise a typical gas sensor that may be modified and coupled to other components, giving it the ability to function in these low oxygen environments. The sensor may operate outside the CA storage room 102. The sensor 120 may be modified to work with the combined air flows by affixing a gas coupler to the electrochemical cell that will contain the sample draw flow coming from the mixing point. The coupler will have an exhaust port so sample air flow will occur parallel to the electrochemical cell diffusion membrane and not allow any ambient air to mix with the sampled air from the mixing point.

Some embodiments of the disclosure may comprise a gas sensor system comprising a first air flow from the interior of a low oxygen storage room; a second air flow from the exterior of the low oxygen storage room, wherein the oxygen content of the second air flow is greater than the oxygen content of the first air flow; a mixing joint joining the first air flow with the second air flow; a gas sensor configured to receive the joined air flows and configured to detect ammonia levels in the joined air flows, wherein the gas sensor is calibrated for the flow rates of the two air flows.

In an embodiment of the gas sensor system, the second air flow contains an oxygen content of at least 20%. In an embodiment of the gas sensor system, the gas sensor comprises an electrochemical gas sensor. In an embodiment of the gas sensor system, the system may further comprise an air vacuum pump configured to draw the joined air flows to the gas sensor. In an embodiment of the gas sensor system, the system may further comprise a first flow meter and a first variable valve configured to monitor and control the flow rate of the first air flow. In an embodiment of the gas sensor system, the system may further comprise a second flow meter and a second variable valve configured to monitor and control the flow rate of the second air flow. In an embodiment of the gas sensor system, the low oxygen storage room is cooled by an ammonia evaporator cooling coil. In an embodiment of the gas sensor system, the low oxygen storage room is configured to store food products. In an embodiment of the gas sensor system, the low oxygen storage room is configured to store pharmaceutical products. In an embodiment of the gas sensor system, the detection comprises a reduction-oxidation reaction. In an embodiment of the gas sensor system, the gas detector issues an alarm when the ammonia level of the joined air flow is more than approximately 20 ppm. In an embodiment of the gas sensor system, the gas detector issues an alarm when the ammonia level of the joined air flow s more than approximately 50 ppm.

Some embodiments of the disclosure may comprise a method for providing gas detection for a low oxygen storage room, the method comprising drawing a first air flow from the interior of the low oxygen storage room; drawing a second air flow from the exterior of the low oxygen storage room, wherein the oxygen content of the second air flow is greater than the oxygen content of the first air flow; monitoring the flow rates of the first air flow and the second air flow; joining the first air flow and the second air flow; feeding the joined air flows to a gas sensor; detecting the gas content of the combined air flows, wherein the detection comprises a reduction-oxidation reaction.

In an embodiment of the method, the method may further comprise calibrating the gas sensor based on the monitored flow rates of the first air flow and the second air flow. In an embodiment of the method, the method may further comprise controlling the flow rate of the first air flow with a first variable valve. In an embodiment of the method, the method may further comprise controlling the flow rate of the second air flow with a second variable valve. In an embodiment of the method, the method may further comprise cooling the low oxygen storage room by an ammonia evaporator cooling coil. In an embodiment of the method, the second air flow contains an oxygen content of at least 20%. In an embodiment of the method, feeding the joined air flows comprises pumping the air flows via an air vacuum pump to the gas sensor. In an embodiment of the method, detecting the gas content comprises detecting the ammonia content of the joined air flows.

Having described various devices and methods herein, exemplary embodiments or aspects can include, but are not limited to:

In a first embodiment, a gas sensor system may comprise a first air flow conduit from the interior of a low oxygen storage room; a second air flow conduit from the exterior of the low oxygen storage room, wherein the oxygen content of a second air flow through the second air flow conduit is greater than an oxygen content of a first air flow through the first air flow conduit; a mixing joint joining the first air flow conduit with the second air flow conduit and in fluid communication with a joined air flow conduit; and a gas sensor configured to receive a joined air flow through the joined air flow conduit and configured to detect ammonia levels in the joined air flow, wherein the gas sensor is calibrated for the flow rates of the two air flows.

A second embodiment can include the gas sensor system of the first embodiment, wherein the second air flow contains an oxygen content of at least 20%.

A third embodiment can include the gas sensor system of the first or second embodiments, wherein the gas sensor comprises an electrochemical gas sensor.

A fourth embodiment can include the gas sensor system of any of the first to third embodiments, further comprising an air vacuum pump configured to draw the joined air flow to the gas sensor.

A fifth embodiment can include the gas sensor system of any of the first to fourth embodiments, further comprising a first flow meter and a first variable valve configured to monitor and control the flow rate of the first air flow.

A sixth embodiment can include the gas sensor system of any of the first to fifth embodiments, further comprising a second flow meter and a second variable valve configured to monitor and control the flow rate of the second air flow.

A seventh embodiment can include the gas sensor system of any of the first to sixth embodiments, wherein the low oxygen storage room is cooled by an ammonia evaporator cooling coil.

An eighth embodiment can include the gas sensor system of any of the first to seventh embodiments, wherein the low oxygen storage room is configured to store food products.

A ninth embodiment can include the gas sensor system of any of the first to eighth embodiments, wherein the low oxygen storage room is configured to store pharmaceutical products.

A tenth embodiment can include the gas sensor system of any of the first to ninth embodiments, wherein the gas sensor is configured to detect ammonia levels using a reduction-oxidation reaction.

An eleventh embodiment can include the method of the tenth embodiment, wherein the gas sensor is configured to issue an alarm when the ammonia level of the joined air flow is more than approximately 20 ppm.

A twelfth embodiment can include the method of the tenth or eleventh embodiments, wherein the gas sensor is configured to issue an alarm when the ammonia level of the joined air flow is more than approximately 50 ppm.

In a thirteenth embodiment, a method for providing gas detection for a low oxygen storage room may comprise drawing a first air flow from the interior of the low oxygen storage room; drawing a second air flow from the exterior of the low oxygen storage room, w herein the oxygen content of the second air flow is greater than the oxygen content of the first air flow; monitoring the flow rates of the first air flow and the second air flow; joining the first air flow and the second air flow; feeding the joined air flows to a gas sensor; and detecting the gas content of the combined air flows, wherein the detection comprises a reduction-oxidation reaction.

A fourteenth embodiment can include the method of the thirteenth embodiment, further comprising calibrating the gas sensor based on the monitored flow rates of the first air flow and the second air flow.

A fifteenth embodiment can include the method of the thirteenth or fourteenth embodiments, further comprising controlling the flow rate of the first air flow with a first variable valve.

A sixteenth embodiment can include the method of any of the thirteenth to fifteenth embodiments, further comprising controlling the flow rate of the second air flow with a second variable valve.

A seventeenth embodiment can include the method of any of the thirteenth to sixteenth embodiments, further comprising cooling the low oxygen storage room by an ammonia evaporator cooling coil.

An eighteenth embodiment can include the method of any of the thirteenth to seventeenth embodiments, wherein the second air flow contains an oxygen content of at least 20%.

A nineteenth embodiment can include the method of any of the thirteenth to eighteenth embodiments, wherein feeding the joined air flows comprises pumping the air flows via an air vacuum pump to the gas sensor.

A twentieth embodiment can include the method of any of the thirteenth to nineteenth embodiments, wherein detecting the gas content comprises detecting the ammonia content of the joined air flows.

In a twenty-first embodiment, a method for providing gas detection for a controlled atmosphere storage room may comprise drawing a first air flow from the interior of the low atmosphere storage room; drawing a second air flow from the exterior of the low atmosphere storage room, wherein the oxygen content of the second air flow is greater than the oxygen content of the first air flow; monitoring the flow rates of the first air flow and the second air flow; joining the first air flow and the second air flow; feeding the joined air flows to a gas sensor; detecting the gas content of the combined air flows, wherein the detection comprises a reduction-oxidation reaction; and in response to detecting the gas content of the combined air flows, determining the gas content of the interior of the low atmosphere storage room using the monitored flow rates for the first air flow and the second air flow.

A twenty-second embodiment can include the method of the twenty-first embodiment, further comprising activating an alarm when the ammonia level of the joined air flow is more than approximately 20 ppm.

A twenty-third embodiment can include the method of the twenty-first or twenty-second embodiments, further comprising calibrating the gas sensor based on the monitored flow rates of the first air flow and the second air flow.

A twenty-fourth embodiment can include the method of any of the twenty-first to twenty-third embodiments, further comprising cooling the low oxygen storage room by an ammonia evaporator cooling coil.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow that scope including all equivalents of the subject matter of the claims. Each and every claim is incorporated as further disclosure into the specification, and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system, or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component,whether electrically, mechanically, or otherwise. Other examples of changes, substitutions, and alterations are ascertainable by one skilled in the art and could be made without departing from the spirit and scope disclosed herein.

1. A gas sensor system comprising:
a first air flow conduit from the interior of a low oxygen storage room;
a second air flow conduit from the exterior of the low oxygen storage room, wherein the oxygen content of a second air flow through the second air flow conduit is greater than an oxygen content of a first air flow through the first air flow conduit;
a mixing joint joining the first air flow conduit with the second air flow conduit and in fluid communication with a joined air flow conduit; and
a gas sensor configured to receive a joined air flow through the joined air flow conduit and configured to detect ammonia levels in the joined air flow, wherein the gas sensor is calibrated for the flow rates of the two air flows.

2. The gas sensor system of claim 1, wherein the second air flow contains an oxygen content of at least 20%.

3. The gas sensor system of claim 1, further comprising an air vacuum pump configured to draw the joined air flow to the gas sensor.

4. The gas sensor system of claim 1, further comprising a first flow meter and a first variable valve configured to monitor and control the flow rate of the first air flow.

5. The gas sensor system of claim 1, further comprising a second flow meter and a second variable valve configured to monitor and control the flow rate of the second air flow.

6. The gas sensor system of claim 1, wherein the low oxygen storage room is cooled by an ammonia evaporator cooling coil.

7. The gas sensor system of claim 1, wherein the gas sensor is configured to detect ammonia levels using a reduction-oxidation reaction.

8. The gas sensor system of claim 1, wherein the gas sensor is configured to issue an alarm when the ammonia level of the joined air flow is more than approximately 20 ppm.

9. The gas sensor system of claim 1, wherein the gas sensor is configured to issue an alarm when the ammonia level of the joined air flow is more than approximately 50 ppm.

10. A method for providing gas detection for a low oxygen storage room, the method comprising:
drawing a first air flow from the interior of the low oxygen storage room;
drawing a second air flow from the exterior of the low oxygen storage room, wherein the oxygen content of the second air flow is greater than the oxygen content of the first air flow;
monitoring the flow rates of the first air flow and the second air flow;
joining the first air flow and the second air flow;
feeding the joined air flows to a gas sensor; and
detecting the gas content of the combined air flows, wherein the detection comprises a reduction-oxidation reaction.

11. The method of claim 10, further comprising calibrating the gas sensor based on the monitored flow rates of the first air flow and the second air flow.

12. The method of claim 10, further comprising controlling the flow rate of the first air flow with a first variable valve.

13. The method of claim 10, further comprising controlling the flow rate of the second air flow with a second variable valve.

14. The method of claim 10, further comprising cooling the low oxygen storage room by an ammonia evaporator cooling coil.

15. The method of claim 10, wherein the second air flow contains an oxygen content of at least 20%.

16. The method of claim 10, wherein detecting the gas content comprises detecting the ammonia content of the joined air flows.

17. A method for providing gas detection for a controlled atmosphere storage room, the method comprising:
drawing a first air flow from the interior of the low atmosphere storage room;
drawing a second air flow from the exterior of the low atmosphere storage room, wherein the oxygen content of the second air flow is greater than the oxygen content of the first air flow;
monitoring the flow rates of the first air flow and the second air flow;
joining the first air flow and the second air flow;
feeding the joined air flows to a gas sensor;
detecting the gas content of the combined air flows, wherein the detection comprises a reduction-oxidation reaction; and
in response to detecting the gas content of the combined air flows, determining the gas content of the interior of the low atmosphere storage room using the monitored flow rates for the first air flow and the second air flow.

18. The method of claim 17, further comprising activating an alarm when the ammonia level of the joined air flow is more than approximately 20 ppm.

19. The method of claim 17, further comprising calibrating the gas sensor based on the monitored flow rates of the first air flow and the second air flow.

20. The method of claim 17, further comprising cooling the low oxygen storage room by an ammonia evaporator cooling coil.
